(19) United States
(12) Patent Application Publication
(10) Pub. No.: US 2014/0018379 A1
(43) Pub. Date: Jan. 16, 2014

(54) PYRIMIDINE DERIVATIVES

(71) Applicants: Roger Tung, Lexington, MA (US); Craig E. Masse, Cambridge, MA (US)

(72) Inventors: Roger Tung, Lexington, MA (US); Craig E. Masse, Cambridge, MA (US)

(73) Assignee: CONCERT PHARMACEUTICALS INC., Lexington, MA (US)

(21) Appl. No.: 13/579,528

(22) PCT Filed: Feb. 18, 2011
(86) PCT No.: PCT/US11/25472

§ 371 Date: Sep. 30, 2013

Related U.S. Application Data
(60) Provisional application No. 61/305,684, filed on Feb. 18, 2010.
(60) Provisional application No. 61/371,814, filed on Aug. 9, 2010.

(51) Int. Cl. C07D 239/48

(52) U.S. Cl. CPC C07D 239/48
USPC 514/275; 544/323; 435/238

(57) ABSTRACT

The present invention provides a compound of Formula I or Formula VI:

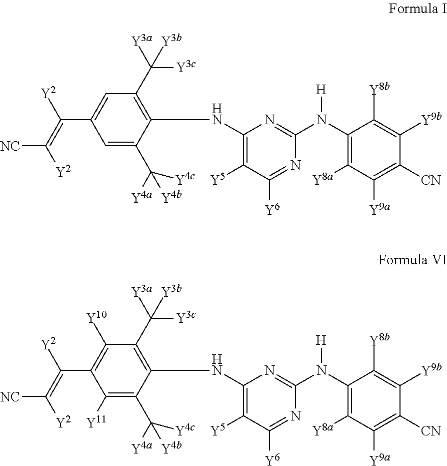

as defined herein. The invention is also directed to compositions comprising the compound of Formula I or Formula VI and methods of therapeutic treatment using the compound of Formula I or Formula VI.

### CROSS-REFERENCE TO RELATED APPLICATIONS

This application claims priority under 35 U.S.C. §119(e) to U.S. Provisional Application No. 61/305,684, filed Feb. 18, 2010, and U.S. Provisional Application No. 61/371,814, filed Aug. 9, 2010, both of which are incorporated by reference herein in their entirety.

### BACKGROUND OF THE INVENTION

This invention relates to novel pyrimidine derivatives and pharmaceutically acceptable salts thereof. This invention also provides compositions comprising a compound of this invention and the use of such compositions in methods of treating diseases and conditions that are beneficially treated by administering a non-nucleoside reverse transcriptase inhibitor (NNRTI).

Rilpivirine, also known as 4-[4-[4-[(E)-2-cyanovinyl]-2,6-dimethylphenylamino]pyrimidin-2-ylamino]benzonitrile hydrochloride, is a known NNRTI. Reverse transcriptase is a crucial enzyme for retroviral replication. Rilpivirine, as an NNRTI, acts by occupying a binding site near the polymerase active site of reverse transcriptase thus distorting and deactivating the nearby polymerase active site and inhibiting the conversion of single-stranded genomic RNA to double-stranded viral DNA.

Rilpivirine is currently undergoing clinical trials for the treatment of HIV.

Results of clinical trials show that rilpivirine is safe and well-tolerated. Headache is the most commonly reported adverse event (12th Conf Retroviruses Opportunistic Infect (CROI) (February 22-25, Boston) 2005, Abst 160).

Despite the beneficial activities of rilpivirine, there is a continuing need for new compounds to treat the aforementioned diseases and conditions.

### DEFINITIONS

The term “treat” means decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of rilpivirine will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66:15; Gannes, L Z et al., Comp Biochem Physiol Mol Integr Physiol, 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3. (99.5% deuterium incorporation).

The term “isotopologue” refers to a species in which the chemical structure differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in tow will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfate, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Stereoisomer” refers to both enantiomers and diastereomers. “Tert” and “t-” each refer to tertiary. “US” refers to the United States of America.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula I:

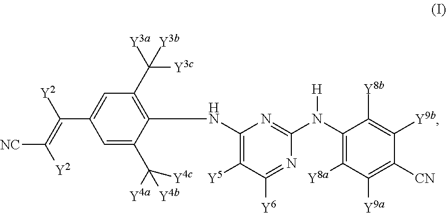

or a pharmaceutically acceptable salt thereof,
wherein each Y is independently hydrogen or deuterium,
provided that at least one Y is deuterium.

In one embodiment Y^{3a}, Y^{3b} and Y^{3c} are the same. In one aspect of this embodiment, each of Y^{3a}, Y^{3b} and Y^{3c} is hydrogen. In another aspect, each of Y^{3a}, Y^{3b} and Y^{3c} is deuterium. In another aspect, each of Y^{4a}, Y^{4b} and Y^{4c} is the same.

In one embodiment Y^{4a}, Y^{4b} and Y^{4c} are the same. In one aspect, each of Y^{4a}, Y^{4b} and Y^{4c} is hydrogen. In another aspect, each of Y^{4a}, Y^{4b} and Y^{4c} is deuterium.

In one embodiment Y¹ and Y² are the same. In one aspect, each of Y¹ and Y² is hydrogen. In another aspect, each of Y¹ and Y² is deuterium.

In one embodiment Y⁵ and Y⁶ are the same. In one aspect, each of Y⁵ and Y⁶ is hydrogen. In another aspect, each of Y⁵ and Y⁶ is deuterium.

In one embodiment Y^{8a} and Y^{8b} are the same. In one aspect, each of Y^{8a} and Y^{8b} is hydrogen. In another aspect, each of Y^{8a} and Y^{8b} is deuterium.

In one embodiment Y^{9a} and Y^{9b} are the same. In one aspect, each of Y^{9a} and Y^{9b} is hydrogen. In another aspect, each of Y^{9a} and Y^{9b} is deuterium.

In a more specific embodiment, Y¹ and Y² are the same; Y^{3a}, Y^{3b}, and Y^{3c} are the same; Y^{4a}, Y^{4b}, and Y^{4c} are the same; Y⁵ and Y⁶ are the same; Y^{8a} and Y^{8b} are the same; and Y^{9a} and Y^{9b} are the same. In one aspect of this embodiment, each of Y^{3a}, Y^{3b} and Y^{3c} is hydrogen. In another aspect of this embodiment, each of Y^{3a}, Y^{3b} and Y^{3c} is deuterium. In one aspect of this embodiment, each of Y^{4a}, Y^{4b} and Y^{4c} is hydrogen. In another aspect of this embodiment, each of Y^{4a}, Y^{4b} and Y^{4c} is deuterium. In one aspect of this embodiment, each of Y¹, Y², Y^{3a}, Y^{3b}, Y^{3c}, Y^{4a}, Y^{4b}, Y^{4c}, Y⁵, Y⁶, Y^{8a}, Y^{8b}, Y^{9a} and Y^{9b} is deuterium.

In another set of embodiments, any atom not designated as deuterium in any of the embodiments set forth above is present at its natural isotopic abundance.

Examples of compounds of Formula I wherein each of Y¹ and Y² is hydrogen, each Y³ is the same, each Y⁴ is the same, each Y⁸ is the same, and each Y⁹ is the same are shown in Table 1 below.

TABLE 1
Examples of Compounds of Formula I

| Cmpd | Each of | Each of | | | Each of | Each of |
|---|---|---|---|---|---|---|
| # | Y³ | Y⁴ | Y⁵ | Y⁶ | Y⁸ | Y⁹ |
| 100 | H | D | H | H | H | H |
| 101 | D | D | H | H | H | H |
| 102 | H | H | D | D | H | H |
| 103 | H | D | D | D | H | H |
| 104 | D | D | D | D | H | H |
| 105 | H | H | H | H | D | H |
| 106 | H | D | H | H | D | H |
| 107 | D | D | H | H | D | H |
| 108 | H | H | D | D | D | H |
| 109 | H | D | D | D | D | H |
| 110 | D | D | D | D | D | H |
| 111 | H | H | H | H | H | D |
| 112 | H | D | H | H | H | D |
| 113 | D | D | H | H | H | D |
| 114 | H | H | D | D | H | D |
| 115 | H | D | D | D | H | D |
| 116 | D | D | D | D | H | D |
| 117 | H | H | H | H | D | D |
| 118 | H | D | H | H | D | D |
| 119 | D | D | H | H | D | D |
| 120 | H | H | D | D | D | D |
| 121 | H | D | D | D | D | D |
| 122 | D | D | D | D | D | D |

or a pharmaceutically acceptable salt thereof, wherein any atom not designated as deuterium in any of the compounds set forth above is present at its natural isotopic abundance.

Examples of compounds of Formula I wherein each of Y¹ and Y² is deuterium, each Y³ is the same, each Y⁴ is the same, each Y⁸ is the same, and each Y⁹ is the same are shown in Table 2 below.

TABLE 2
Examples of Compounds of Formula I

| Cmpd | Each of | Each of | | | Each of | Each of |
|---|---|---|---|---|---|---|
| # | Y³ | Y⁴ | Y⁵ | Y⁶ | Y⁸ | Y⁹ |
| 125 | H | D | H | H | H | H |
| 126 | D | D | H | H | H | H |
| 127 | H | H | D | D | H | H |
| 128 | H | D | D | D | H | H |
| 129 | D | D | D | D | H | H |
| 130 | H | H | H | H | D | H |
| 131 | H | D | H | H | D | H |
| 132 | D | D | H | H | D | H |
| 133 | H | H | D | D | D | H |
| 134 | H | D | D | D | D | H |
| 135 | D | D | D | D | D | H |
| 136 | H | H | H | H | H | D |
| 137 | H | D | H | H | H | D |
| 138 | D | D | H | H | H | D |
| 139 | H | H | D | D | H | D |
| 140 | H | D | D | D | H | D |
| 141 | D | D | D | D | H | D |
| 142 | H | H | H | H | D | D |
| 143 | H | D | H | H | D | D |
| 144 | D | D | H | H | D | D |
| 145 | H | H | D | D | D | D |
| 146 | H | D | D | D | D | D |
| 147 | D | D | D | D | D | D |

or a pharmaceutically acceptable salt thereof, wherein any atom not designated as deuterium in any of the compounds set forth above is present at its natural isotopic abundance.

The present invention is also directed to compounds of Formula II-V or salts thereof:

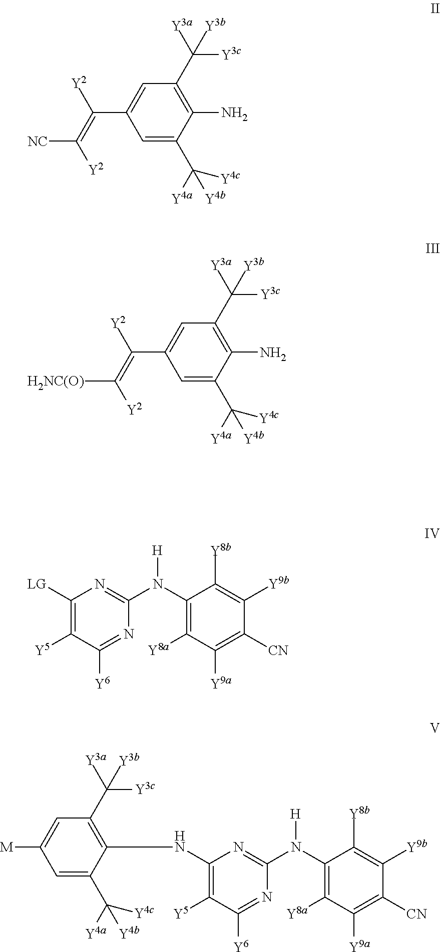

In compounds of Formula IV, LG represents a suitable leaving group. In compounds of Formula V, M represents C(═O)H, C(═O)OC₁-C₆ alkyl such as C(═O)OCH₃, or LG, wherein LG represents a suitable leaving group. Suitable leaving groups LG in compounds of Formula IV and V are known to those skilled in the art as being useful for synthetic coupling to either compounds of Formula II or III. Examples of suitable leaving groups LG in compounds of Formula IV and V include halo; C₁-C₆ alkylsulfonate optionally substituted with halo; C₆-C₁₀ arylsulfonate optionally substituted with alkyl, halo, or a combination thereof; and dialkylphosphate.

The present invention also provides a compound of Formula VI:

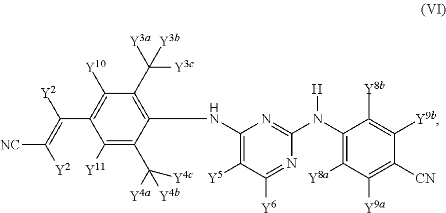

or a pharmaceutically acceptable salt thereof,
wherein each Y is independently hydrogen or deuterium,
provided that at least one Y is deuterium.

In one embodiment Y^{3a}, Y^{3b} and Y^{3c} are the same. In one aspect of this embodiment, each of Y^{3a}, Y^{3b} and Y^{3c} is hydrogen. In another aspect, each of Y^{3a}, Y^{3b} and Y^{3c} is deuterium. In another aspect, each of Y^{4a}, Y^{4b} and Y^{4c} is the same.

In one embodiment Y^{4a}, Y^{4b} and Y^{4c} are the same. In one aspect, each of Y^{4a}, Y^{4b} and Y^{4c} is hydrogen. In another aspect, each of Y^{4a}, Y^{4b} and Y^{4c} is deuterium.

In one embodiment Y¹ and Y² are the same. In one aspect, each of Y¹ and Y² is hydrogen. In another aspect, each of Y¹ and Y² is deuterium.

In one embodiment Y⁵ and Y⁶ are the same. In one aspect, each of Y⁵ and Y⁶ is hydrogen. In another aspect, each of Y⁵ and Y⁶ is deuterium.

In one embodiment Y^{8a} and Y^{8b} are the same. In one aspect, each of Y^{8a} and Y^{8b} is hydrogen. In another aspect, each of Y^{8a} and Y^{8b} is deuterium.

In one embodiment Y^{9a} and Y^{9b} are the same. In one aspect, each of Y^{9a} and Y^{9b} is hydrogen. In another aspect, each of Y^{9a} and Y^{9b} is deuterium.

In one embodiment Y¹⁰ and Y¹¹ are the same. In one aspect, each of Y¹⁰ and Y¹¹ is hydrogen. In another aspect, each of Y¹⁰ and Y¹¹ is deuterium.

In a more specific embodiment, Y¹ and Y² are the same; Y^{3a}, Y^{3b}, and Y^{3c} are the same; Y^{4a}, Y^{4b}, and Y^{4c} are the same; Y⁵ and Y⁶ are the same; Y^{8a} and Y^{8b} are the same; Y^{9a} and Y^{9b} are the same; and Y¹⁰ and Y¹¹ are the same. In one aspect of this embodiment, each of Y^{3a}, Y^{3b} and Y^{3c} is hydrogen. In another aspect of this embodiment, each of Y^{3a}, Y^{3b} and Y^{3c} is deuterium. In one aspect of this embodiment, each of Y^{4a}, Y^{4b} and Y^{4c} is hydrogen. In another aspect of this embodiment, each of Y^{4a}, Y^{4b} and Y^{4c} is deuterium. In one aspect of this embodiment, each of Y¹, Y², Y^{3a}, Y^{3b}, Y^{3c}, Y^{4a}, Y^{4b}, Y^{4c}, Y⁵, Y⁶, Y^{8a}, Y^{8b}, Y^{9a}, Y^{9b}, Y¹⁰ and Y¹¹ is deuterium.

In another set of embodiments, any atom not designated as deuterium in any of the embodiments set forth above is present at its natural isotopic abundance.

Examples of compounds of Formula VI wherein each of Y¹ and Y² is hydrogen, each of Y¹⁰ and Y¹¹ is deuterium, each Y³ is the same, each Y⁴ is the same, each Y⁸ is the same, and each Y⁹ is the same are shown in Table 3 below.

TABLE 3
Examples of Compounds of Formula VI

| Cmpd | Each of | Each of | | | Each of | Each of |
|---|---|---|---|---|---|---|
| # | Y³ | Y⁴ | Y⁵ | Y⁶ | Y⁸ | Y⁹ |
| 200 | H | D | H | H | H | H |
| 201 | D | D | H | H | H | H |
| 202 | H | H | D | D | H | H |
| 203 | H | D | D | D | H | H |
| 204 | D | D | D | D | H | H |
| 205 | H | H | H | H | D | H |
| 206 | H | D | H | H | D | H |
| 207 | D | D | H | H | D | H |
| 208 | H | H | D | D | D | H |
| 209 | H | D | D | D | D | H |
| 210 | D | D | D | D | D | H |
| 211 | H | H | H | H | H | D |
| 212 | H | D | H | H | H | D |
| 213 | D | D | H | H | H | D |
| 214 | H | H | D | D | H | D |
| 215 | H | D | D | D | H | D |
| 216 | D | D | D | D | H | D |
| 217 | H | H | H | H | D | D |
| 218 | H | D | H | H | D | D |
| 219 | D | D | H | H | D | D |
| 220 | H | H | D | D | D | D |
| 221 | H | D | D | D | D | D |
| 222 | D | D | D | D | D | D |

or a pharmaceutically acceptable salt thereof, wherein any atom not designated as deuterium in any of the compounds set forth above is present at its natural isotopic abundance.

Examples of compounds of Formula VI wherein each of Y¹ and Y² is deuterium, each of Y¹⁰ and Y¹¹ is deuterium, each Y³ is the same, each Y⁴ is the same, each Y⁸ is the same, and each Y⁹ is the same are shown in Table 4 below.

TABLE 4
Examples of Compounds of Formula VI

| Cmpd | Each of | Each of | | | Each of | Each of |
|---|---|---|---|---|---|---|
| # | Y³ | Y⁴ | Y⁵ | Y⁶ | Y⁸ | Y⁹ |
| 225 | H | D | H | H | H | H |
| 226 | D | D | H | H | H | H |
| 227 | H | H | D | D | H | H |
| 228 | H | D | D | D | H | H |
| 229 | D | D | D | D | H | H |
| 230 | H | H | H | H | D | H |
| 231 | H | D | H | H | D | H |
| 232 | D | D | H | H | D | H |
| 233 | H | H | D | D | D | H |
| 234 | H | D | D | D | D | H |
| 235 | D | D | D | D | D | H |
| 236 | H | H | H | H | H | D |
| 237 | H | D | H | H | H | D |
| 238 | D | D | H | H | H | D |
| 239 | H | H | D | D | H | D |
| 240 | H | D | D | D | H | D |
| 241 | D | D | D | D | H | D |
| 242 | H | H | H | H | D | D |
| 243 | H | D | H | H | D | D |
| 244 | D | D | H | H | D | D |
| 245 | H | H | D | D | D | D |
| 246 | H | D | D | D | D | D |
| 247 | D | D | D | D | D | D |

or a pharmaceutically acceptable salt thereof, wherein any atom not designated as deuterium in any of the compounds set forth above is present at its natural isotopic abundance.

The present invention is also directed to compounds of Formula VII-IX or salts thereof:

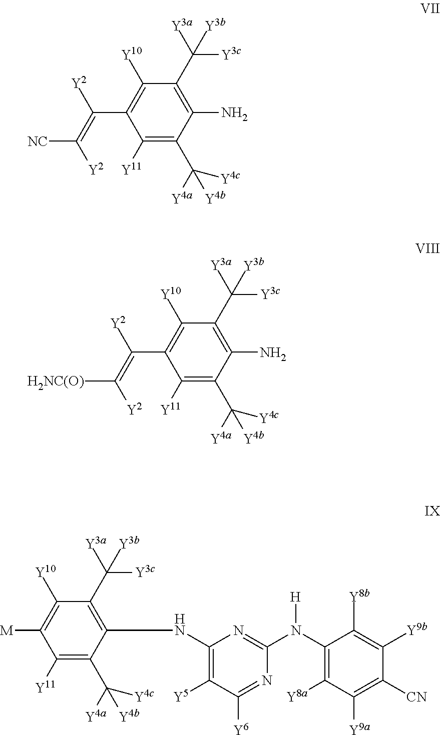

In compounds of Formula IX, M represents C(═O)H, C(═O)OC₁-C₆ alkyl such as C(═O)OCH₃, or LG, wherein LG represents a suitable leaving group. Suitable leaving groups LG in compounds of Formula IX are known to those skilled in the art as being useful for synthetic coupling to yield compounds of Formula VI. Examples of suitable leaving groups LG in compounds of Formula IX include halo; C₁-C₆ alkylsulfonate optionally substituted with halo; C₆-C₁₀ arylsulfonate optionally substituted with alkyl, halo, or a combination thereof; and dialkylphosphate.

### Synthesis

Compounds of Formula I or Formula VI may be conveniently prepared in a manner analogous to the one described in U.S. Pat. No. 7,125,879 by replacing the reagents and/or starting materials described in the schemes and the examples in the '879 patent with suitable deuterated counterparts to obtain derivatives of rilpivirine having the deuteration patterns disclosed herein. For example, the deuterated counterparts of the reagents and/or starting materials described in the schemes and examples of the '879 patent may be commercially available deuterated compounds. The schemes of the '879 patent, shown on column 14, line 23-column 41, line 56, and the examples of the '879 patent, shown on column 51, line 6-column 119, line 19, are incorporated by reference herein. As another example, the Compounds of Formula I or Formula VI may be conveniently prepared in a manner analogous to the one described in Guillemont et al., J. Med. Chem. 2005, 48, 2072-2079, by replacing the reagents and/or starting materials described in Guillemont et al. with suitable deuterated counterparts to obtain derivatives of rilpivirine having the deuteration patterns disclosed herein. For example, the deuterated counterparts of the reagents and/or starting materials described in the schemes and examples of the J. Med. Chem. 2005 reference may be commercially available deuterated compounds. Guillemont et al. is incorporated by reference herein in its entirety. Such approaches are not intended to be limiting.

A convenient method for synthesizing compounds of Formula I or Formula VI is depicted in Scheme 1.

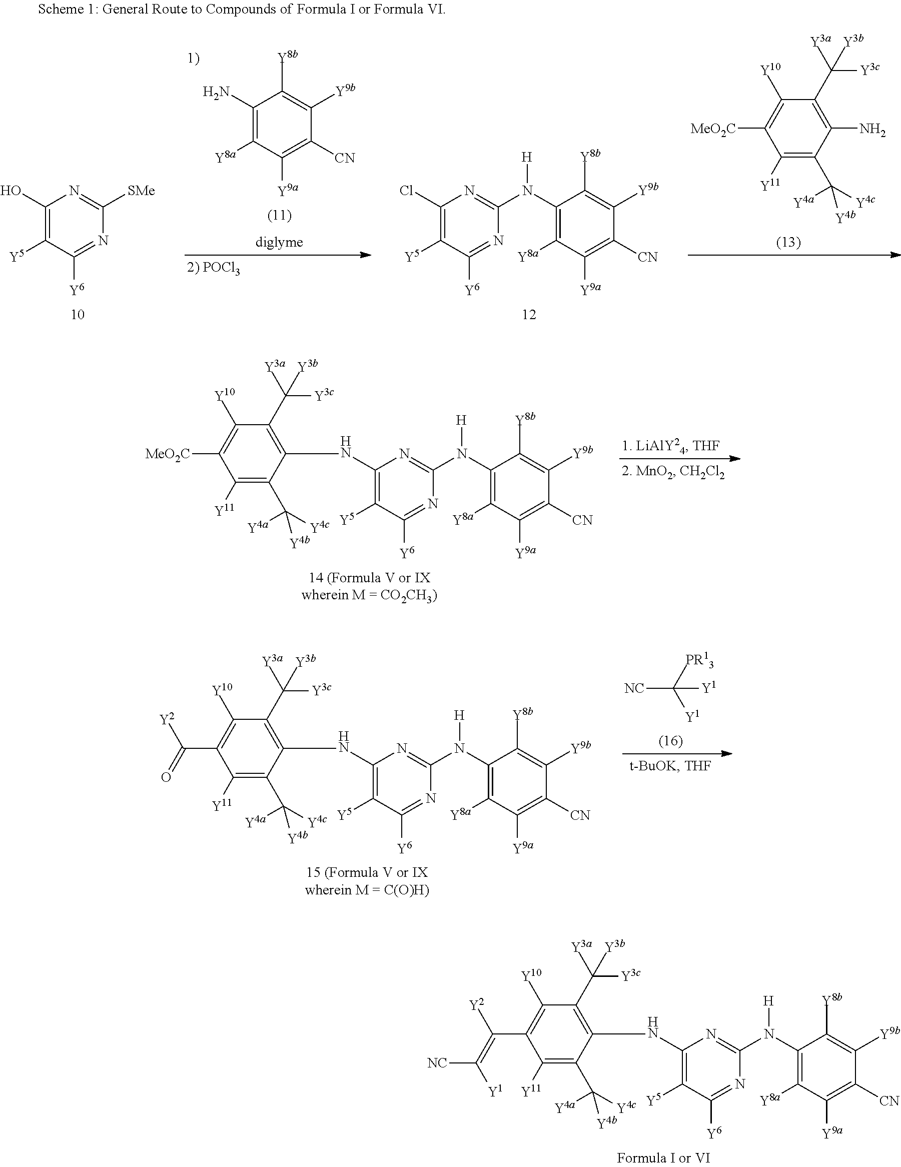

Scheme 1 depicts a general route to compounds of Formula I or Formula VI following the general methods of Guillemont, J. et al. J. Med. Chem. 2005, 48(6), 2072-2079; De Corte, B. L. et al. Bioorg. Med. Chem. Lett. 2001, 11, 2235-2239; and the '879 patent. Appropriately-deuterated intermediate 12 may be prepared through the coupling of appropriately-deuterated thioether 10 with appropriately-deuterated 11 followed by chlorination with POCl₃. Coupling of 12 with appropriately-deuterated aniline 13 affords intermediate 14. Reduction of the methyl ester with LiAlY²₄ (Y²=H or D) followed by oxidation of the resulting alcohol affords aldehyde 15. Wittig or Horner-Wadsworth-Emmons coupling with nitrile 16, wherein R¹ may be, for example, a C₁-C₆ alkyl such as methyl or ethyl, or a C₆-C₁₀ aryl such as phenyl, affords compounds of Formula I or Formula VI. One skilled in the art will appreciate that deuterated solvents and reagents may be substituted, where appropriate, to afford compounds of Formula I or Formula VI bearing different patterns of deuterium substitution.

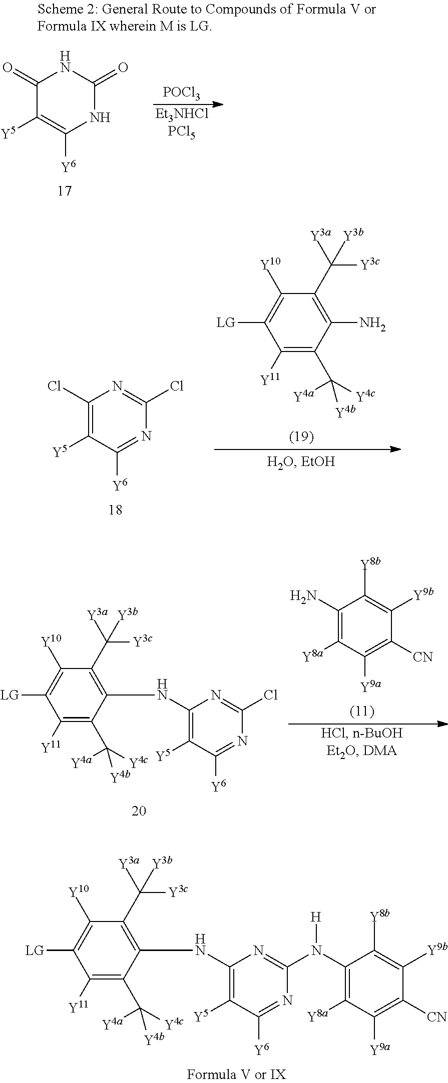

Scheme 2 depicts an alternative route to a compound of Formula V or Formula IX wherein M is LG following the general methods of patents GB 2287466, WO 2005028479, and WO 2007085833. Treatment of appropriately-deuterated commercially-available intermediate 17 with POCl₃ affords appropriately-deuterated 18, as described in GB 2287466. Conversion of 18 to 20 may be effected by treatment of 18 with an appropriately-deuterated 2,6-dimethyl aniline intermediate 19 under conditions analogous to those described in WO 2005028479. Treatment of 20 with appropriately deuterated aniline 11 affords a compound of Formula V or Formula IX wherein M is LG under conditions analogous to those described in WO 2007085833. One skilled in the art will appreciate that deuterated solvents and reagents may be substituted, where appropriate, to afford compounds of Formula V or Formula IX wherein M is LG bearing different patterns of deuterium substitution.

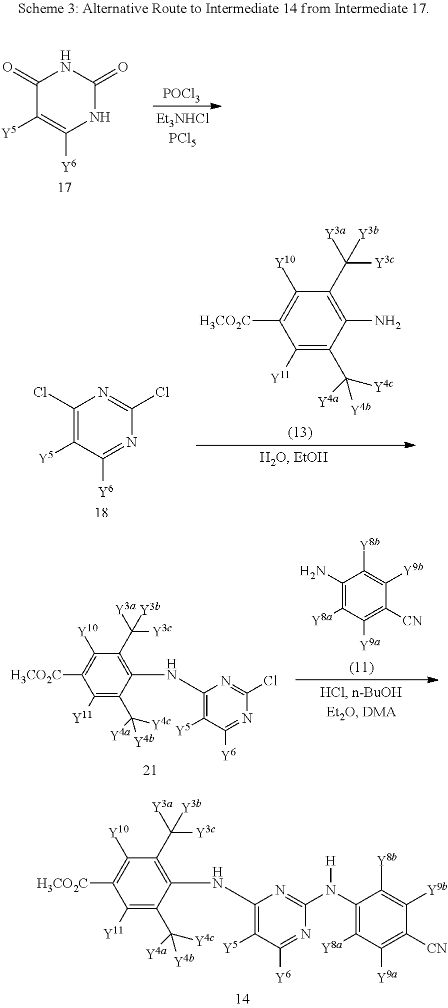

Scheme 3 depicts a route to intermediate 14 alternative to that shown in Scheme 1 following the general methods of patents GB 2287466, WO 2005028479, and WO 2007085833. Treatment of appropriately-deuterated intermediate 17 (commercially available forms include those with Y⁵=Y⁶=D; Y⁵=H and Y⁶=D; and Y⁵=D and Y⁶=D) with POCl₃ affords appropriately-deuterated 18, in a manner analogous to that described in GB 2287466. Conversion of 18 to 21 may be effected by treatment of 18 with intermediate 13 under conditions analogous to those described in WO 2005028479. Treatment of 21 with appropriately deuterated aniline 11 under conditions analogous to those described in WO 2007085833 affords intermediate 14. One skilled in the art will appreciate that deuterated solvents and reagents may be substituted, where appropriate, to afford embodiments of 14 bearing different patterns of deuterium substitution.

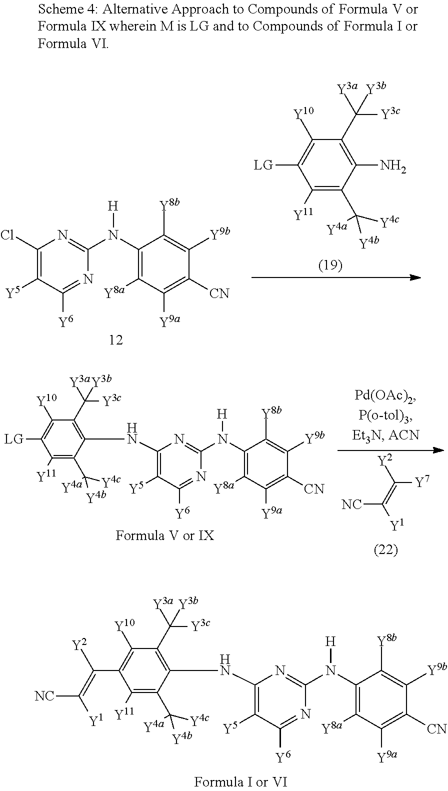

Scheme 4 depicts an alternative route to compounds of Formula I or Formula VI, following the general methods of Guillemont, J. J. Med. Chem. 2005, 48(6), 2072-2079 and the '879 patent. Coupling of 12 with appropriately-deuterated aniline 19 affords compounds of Formula V or Formula IX wherein M is LG. Palladium-mediated coupling with nitrile 22 (Y⁷=H, D or metal) affords compounds of Formula I or Formula VI.

A convenient method for synthesizing compounds of Formula II or Formula VII is depicted in Scheme 5.

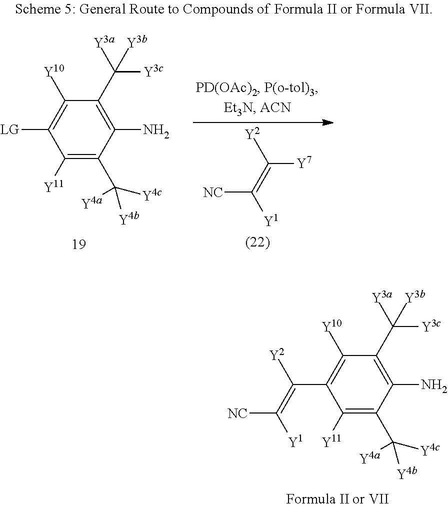

Scheme 5 depicts a general route to compounds of Formula II or Formula VII. Following conditions analogous to those previously described in Guillemont, J. J. Med. Chem. 2005, 48(6), 2072-2079, palladium-mediated coupling of 19 with nitrile 22 (Y⁷=H, D or metal) affords compounds of Formula II or Formula VII.

A convenient method for synthesizing compounds of Formula III or Formula VIII is depicted in Scheme 6.

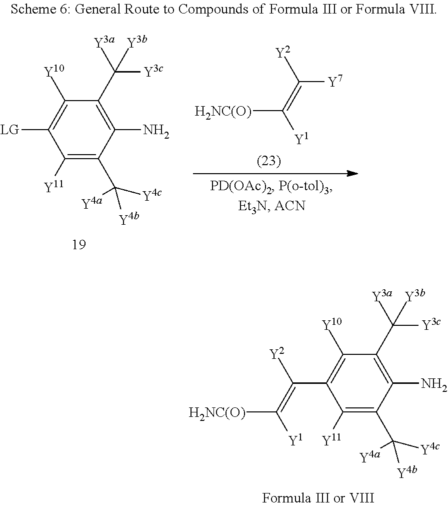

Scheme 6 depicts a general route to compounds of Formula III or Formula VIII. Under conditions analogous to those previously described in the '879 patent, palladium-mediated coupling of 19 with acrylamide 23 (Y⁷=H, D or metal) affords compounds of Formula III or Formula VIII.

A convenient method for synthesizing compounds of Formula IV is depicted in Scheme 7.

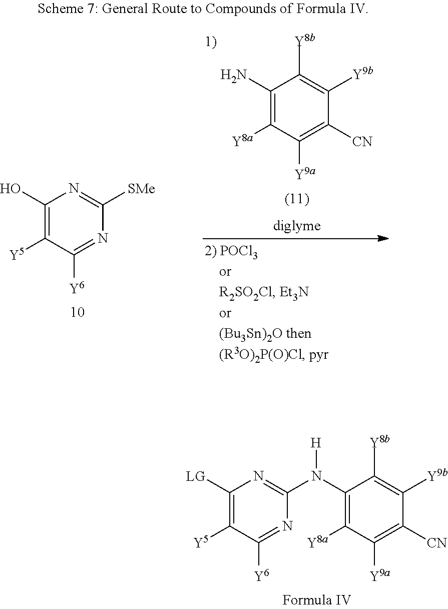

Scheme 7 depicts a general route to compounds of Formula IV. Coupling of appropriately-deuterated thioether 10 with appropriately-deuterated 11 followed by activation of the 4-hydroxyl affords compounds of Formula IV. In Scheme 7, R² in R²SO₂Cl may be, for example, C₁-C₆ alkyl such as methyl or ethyl, and R³ in (R³O)₂P(O)Cl may be, for example, C₁-C₆ alkyl such as methyl or ethyl. Possible activation conditions include POCl₃ (De Corte, B. L. et al. Bioorg. Med. Chem. Lett. 2001, 11, 2235-2239), C₁-C₆ alkylsulfonyl chlorides or C₆-C₁₀ arylsulfonyl chlorides in the presence of base (Zhang, H. et al. Bioorg. Med. Chem. Lett. 2010, 20(1), 60-64), and stannylation of the 4-hydroxyl with (Bu₃Sn)₂O followed by treatment with dialkyl chlorophosphates (Tanimura, H. Tetrahedron Lett. 1986, 27(34), 4047-50).

The following intermediates, of potential interest for use in Schemes 1, 2, 3, 4, 5, 6 and 7 above, are commercially available:

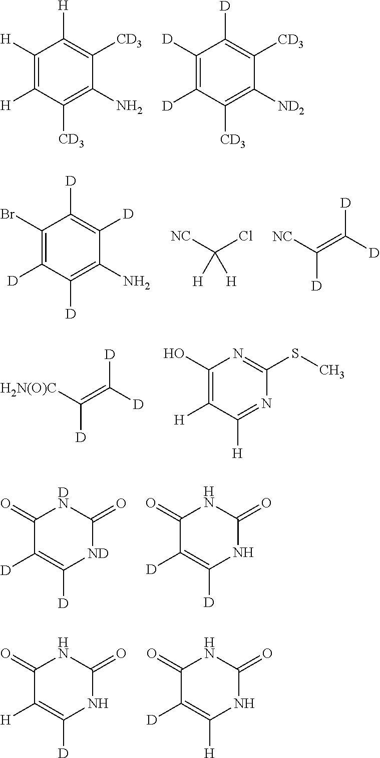

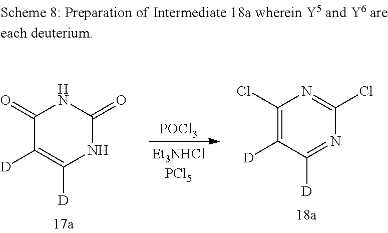

Scheme 8 depicts a possible route to 18a following a method analogous to that of GB patent publication 2287466. Treatment of deuterated commercially-available intermediate 17a with POCl₃ affords deuterated 18a.

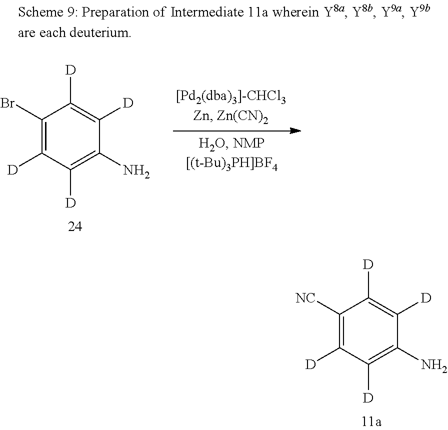 [SMILES: [2H]c1c([2H])c(Br)c([2H])c([2H])c1N ; [2H]c1c([2H])c(C#N)c([2H])c([2H])c1N]

Scheme 9 depicts a possible route to 11a following a method analogous to that of Stazi, F., et al. Tetrahedron Lett. 2005, 46(11), 1815-1818. Palladium-mediated cyanation of commercially-available bromide 24 with Zn(CN)₂ affords nitrile 11a.

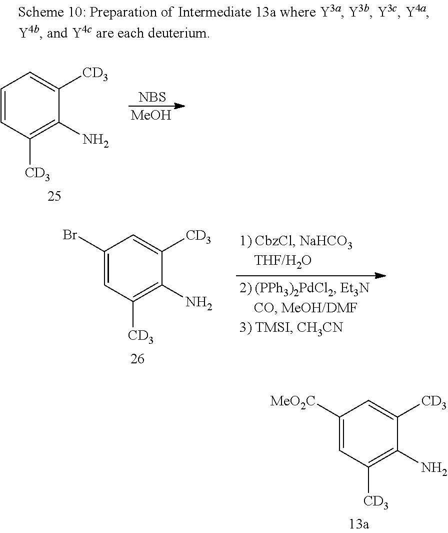

Scheme 10 depicts a possible route for the preparation of aniline 13a following the general methods of Rajagopal, R. et al. J. Mol. Catal. A: Chem. 2004, 210(1-2), 165-169 and Corbett, J. W. et al. Bioorg. Med. Chem. Lett. 2007, 17(24), 6707-6713. Commercially-available aniline 25 may be brominated with NBS to afford bromide 26. 26 may be converted to 13a by a three-step procedure consisting of protection of the amino moiety as the carbobenzyloxy carbamate, palladium-mediated carbomethoxylation, and cleavage of the carbamate moiety with TMSI. Intermediate 13b may be prepared according to the same procedure with the exception that 2,6-dimethylaniline-d11 is used in place of starting compound 25.

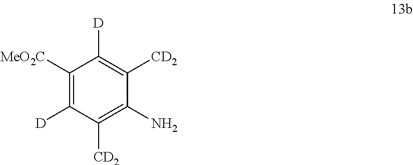

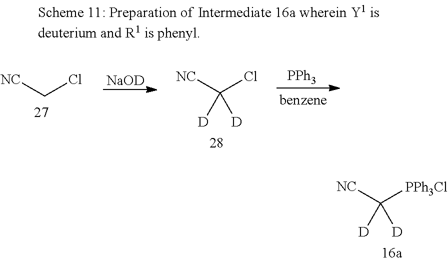

Scheme 11 depicts a possible route for the preparation of Wittig reagent 16a following the general methods of Take, L. et al. Liebigs Ann. Chem. 1982, 924-929 and Abramovitch, R. A. et al. J. Org. Chem. 1980, 45(26), 5316-5319. Commercially-available chloroacetonitrile 27 may be deuterated with sodium deuteroxide to afford 28. Displacement of the chloride with PPh₃ affords 16a.

Additional methods of synthesizing compounds of Formula I or Formula VI and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene, T W et al., Protective Groups in Organic Synthesis, 3^{rd} Ed., John Wiley and Sons (1999); Fieser, L et al., Fieser and Fieser's Reagents for Organic Synthesis, John Wiley and Sons (1994); and Paquette, L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free pharmaceutical compositions comprising an effective amount of a compound of Formula I or Formula VI (e.g., including any of the formulae herein) or a pharmaceutically acceptable salt thereof; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the subject, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as rilpivirine.

Preferably, the second therapeutic agent is an agent useful in the treatment of HIV infection, such as an infection related to HIV-1.

In one embodiment, the second therapeutic agent can be selected from one or more of a HIV protease inhibitor (e.g., amprenavir, fosamprenavir, tipranavir, indinavir, saquinavir, lopinavir, ritonavir, atazanavir, or nelfinavir); a second non-nucleoside reverse transcriptase inhibitor (“NNRTI”) (e.g., UK-453061, GSK 2248761, etravirine, delavirdine, efavirenz, or nevirapine); a nucleoside/nucleotide reverse transcriptase inhibitor (“NRTI”) (e.g., zidovudine, lamivudine, emtricitabine, tenofovir disoproxil fumarate, didanosine, stavudine, abacavir, racivir, amdoxovir, apricitabine, entecavir, adefovir or elvucitabine); a CCR5 antagonist (e.g., PF-232798; GSK 706769, enfuvirtide, maraviroc, vicriviroc, PRO 140, or TNX-355); an integrase inhibitor (e.g., GSK 1349572, raltegravir, or elvitegravir); an immune based antiretroviral agent (e.g., immunitin, proleukin, remune, BAY 50-4798 or IR103); a viral maturation inhibitor (e.g., bevirimat); a cellular inhibitor (e.g., droxia or hydroxyurea); or a pharmacokinetic enhancing agent (e.g., ritonavir, GS 9350; PF-03716539).

In another embodiment, the second therapeutic agent is selected from efavirenz, didanosine, tenofovir disoproxil, nelfinavir mesylate, raltegravir, saquinavir, lopinavir, nevirapine, emtricitabine, abacavir, lamivudine, zidovudine, maraviroc, stavudine, darunavir, fosamprenavir, vicriviroc, GSK 1349572, UK-453061, PF-03716539, etravirine, pharmaceutically acceptable salts of any of the foregoing, and combinations thereof.

In a further embodiment, the second therapeutic agent is selected from zidovudine, lamovudine, tenofovir, and emtricitabine.

In another embodiment, the invention provides separate dosage forms of a compound of this invention and one or more of any of the above-described second therapeutic agents, wherein the compound or a pharmaceutically acceptable salt thereof and the second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat the target disorder.

The interrelationship of dosages for animals and humans (based on milligrams per meter squared of body surface) is described in Freireich et al., Cancer Chemother. Rep, 1966, 50: 219. Body surface area may be approximately determined from height and weight of the subject. See, e.g., Scientific Tables, Geigy Pharmaceuticals, Ardsley, N.Y., 1970, 537.

In one embodiment, an effective amount of a compound of this invention can range from 0.1 mg to about 3000 mg, such as about 2.5 mg to about 600 mg, such as about 12.5 to about 300 mg, such as from about 25 to about 200 mg, such as from about 50 mg to about 100 mg.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician. For example, guidance for selecting an effective dose can be determined by reference to the prescribing information for rilpivirine.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of inhibiting the conversion of single-stranded genomic RNA to double-stranded viral DNA in a cell, comprising contacting a cell with one or more compounds of Formula I or Formula VI herein or a pharmaceutically acceptable salt thereof. According to another embodiment, the invention provides a method of treating a disease that is beneficially treated by a compound of Formula I or Formula VI or a pharmaceutically acceptable salt thereof, or a composition of the invention, in a subject in need thereof, comprising the step of administering to the subject an effective amount of a compound or a composition of this invention. Such diseases include, but are not limited to, HIV infection.

Identifying a subject in need of such treatment can be in the judgment of a subject or a health care professional and can be subjective (e.g. opinion) or objective (e.g. measurable by a test or diagnostic method).

In another embodiment, any of the above methods of treatment comprises the further step of co-administering to the subject in need thereof one or more second therapeutic agents. The choice of second therapeutic agent may be made from any second therapeutic agent known to be useful for co-administration with rilpivirine. For the treatment of HIV infection, useful second therapeutic agents for co-administration include one or more of a HIV protease inhibitor (e.g., amprenavir, fosamprenavir, tipranavir, indinavir, saquinavir, lopinavir, ritonavir, atazanavir, or nelfinavir); a second non-nucleoside reverse transcriptase inhibitor (“NNRTI”) (e.g., UK-453061, GSK 2248761, etravirine, delavirdine, efavirenz, or nevirapine); a nucleoside/nucleotide reverse transcriptase inhibitor (“NRTI”) (e.g., zidovudine, lamivudine, emtricitabine, tenofovir disoproxil fumarate, didanosine, stavudine, abacavir, racivir, amdoxovir, apricitabine, entecavir, adefovir or elvucitabine); a CCR5 antagonist (e.g., PF-232798; GSK 706769, enfuvirtide, maraviroc, vicriviroc, PRO 140, or TNX-355); an integrase inhibitor (e.g., GSK 1349572, raltegravir, or elvitegravir); an immune based antiretroviral agent (e.g., immunitin, proleukin, remune, BAY 50-4798 or IR103); a viral maturation inhibitor (e.g., bevirimat); a cellular inhibitor (e.g., droxia or hydroxyurea); or a pharmacokinetic enhancing agent (e.g., ritonavir, GS 9350; PF-03716539).

In a more specific embodiment, the combination therapies of this invention include co-administering to a patient in need thereof a compound of Formula I or Formula VI or a pharmaceutically acceptable salt thereof and a second therapeutic agent selected from efavirenz, didanosine, tenofovir disoproxil, nelfinavir mesylate, raltegravir, saquinavir, lopinavir, nevirapine, emtricitabine, abacavir, lamivudine, zidovudine, maraviroc, stavudine, darunavir, fosamprenavir, vicriviroc, GSK 1349572, UK-453061, PF-03716539, etravirine, pharmaceutically acceptable salts of any of the foregoing, and combinations thereof.

In a further specific embodiment, the combination therapies of this invention include co-administering to a patient in need thereof a compound of Formula I or Formula VI or a pharmaceutically acceptable salt thereof and a second therapeutic agent selected from zidovudine, lamovudine, tenofovir, and emtricitabine.

The term “co-administered” as used herein means that the second therapeutic agent may be administered together with a compound of this invention as part of a single dosage form (such as a composition of this invention comprising a compound of the invention and a second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I or Formula VI or a pharmaceutically acceptable salt thereof alone or together with one or more of the above-described second therapeutic agents in the manufacture of a medicament, either as a single composition or as separate dosage forms, for treatment in a subject of a disease, disorder or symptom set forth above.

Another aspect of the invention is a compound of Formula I or Formula VI or a pharmaceutically acceptable salt thereof for use in the treatment in a subject of a disease, disorder or symptom thereof delineated herein.

### EXAMPLES

### Example 1

### Synthesis of 4-Amino-2,3,5,6-d₄-benzonitrile (11a)

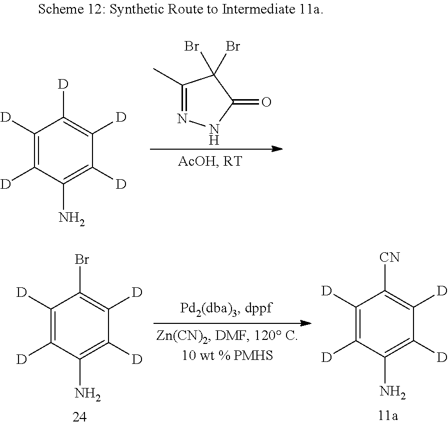

### Step 1. 4-Bromo-2,3,5,6-d₄-aniline (24)

To a solution of aniline-d5 (5.30 g, 54.1 mmol, Aldrich, 98 atom % D) in acetic acid (170 mL) was added 4,4-dibromo-3-methyl-2-pyrazolin-5-one (13.8 g, 54.1 mmol) portionwise over 30 minutes. The reaction stirred at room temperature for 15 hours and the precipitate was removed via filtration rinsing with cold acetic acid. The acetic acid solution was then diluted with water and brought to pH>7 via careful addition of solid K₂CO₃. The resulting solution was extracted with 1:1 Et₂O/heptane (3×100 mL) and the organic layers were combined, washed with water, dried (MgSO₄), filtered and concentrated under reduced pressure. The resulting residue was purified via silica gel column chromatography (0-30% EtOAc/heptane) to afford 4-bromo-2,3,5,6-tetradeuteroaniline (24) as a tan solid (5.67 g, 60%). MS (ESI) 176.1 [(M+H)⁺].

### Step 2. 4-Amino-2,3,5,6-d₄-benzonitrile (11a)

To a solution of 24 (5.67 g, 32.2 mmol) in DMF (50 mL) was added Zn(CN)₂ (4.54 g, 38.7 mmol) followed by PMHS (560 μL), Pd₂(dba)₃ (1.47 g, 1.61 mmol), dppf (893 mg, 1.61 mmol) and water (1.40 mL). The reaction stirred at 120° C. for 15 hours then was cooled to room temperature, diluted with Et₂O (˜250 mL) and filtered through a silica plug rinsing with EtOAc. The resulting solution was diluted with water, extracted with Et₂O (5×75 mL), and the organic layers were combined, washed with brine, dried (MgSO₄), filtered and concentrated under reduced pressure. The resulting residue was purified via silica gel column chromatography (0-30% EtOAc/heptane) to afford 4-amino-2,3,5,6-tetradeuterobenzonitrile (11a) as a tan solid (3.09 g, 79%). MS (ESI) 123.1 [(M+H)⁺].

### Example 2

### Synthesis of 4-Bromo-3,5-d₂-2,6-bis d₃-methyl)aniline (6)

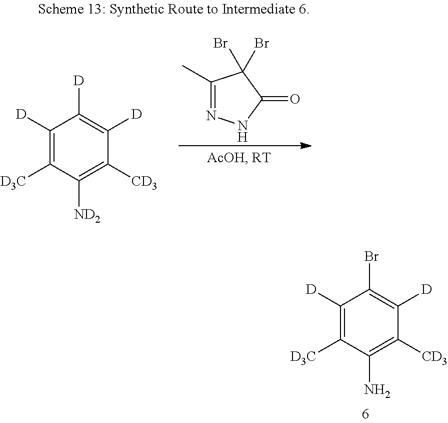

### 4-Bromo-3,5-d₂-2,6-bis(d₃-methyl)aniline (6)

Prepared following the procedure described for the synthesis of 4-bromo-2,3,5,6-tetradeuteroaniline (24) employing 2,6-dimethylaniline-d11 (955 mg, 7.23 mmol, CDN Isotopes, 98 atom % D) to afford 4-bromo-3,5-dideutero-2,6-bis(trideuteromethyl)aniline (6) as a tan solid (1.27 g, 84%). MS (ESI) 208.1 [(M+H)⁺].

### Example 3

### Synthesis of (E)-2,3,5,6-d₄-4-((4-3,5-d₂-2-cyanovinyl)-2,6-bis(d₃-methyl)phenyl)amino)pyrimidin-2-yl)amino)benzonitrile (Compound 244)

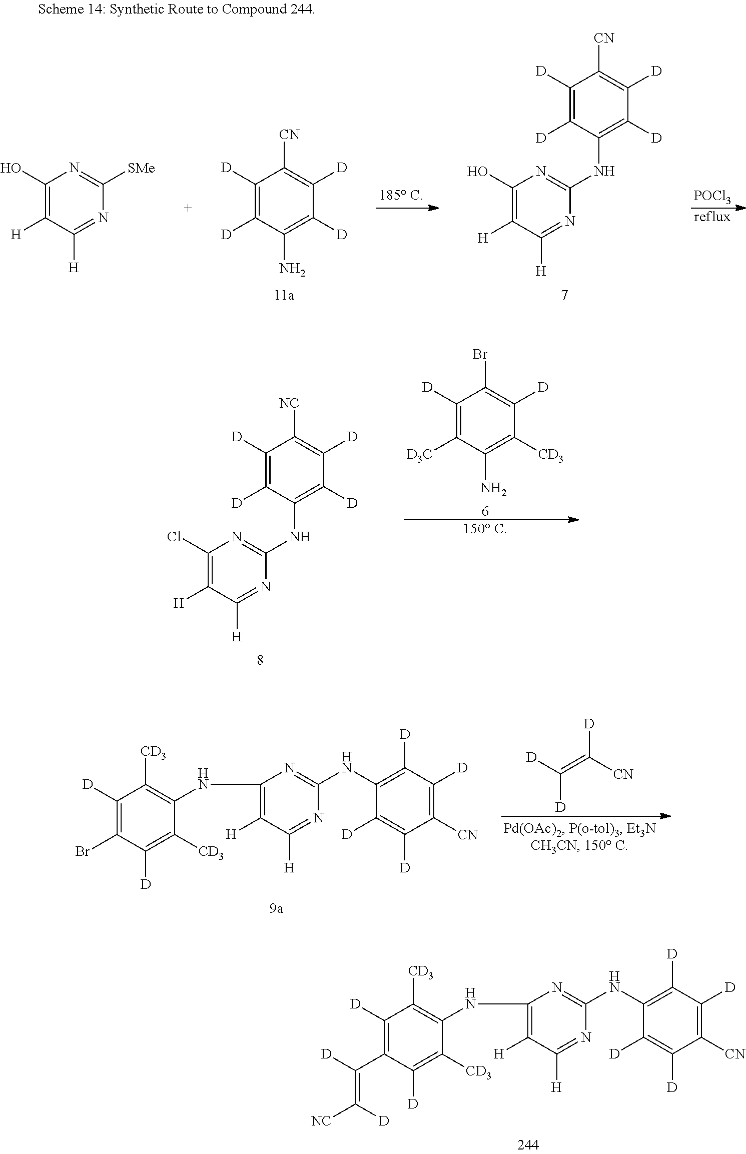

### Step 1. 2,3,5,6-Tetradeutero-4-((4-hydroxypyrimidin-2-yl)amino)benzonitrile (7)

A mixture of 2-(methylthio)pyrimidin-4(1H)-one (466 mg, 3.28 mmol) and 11a (1.00 g, 8.20 mmol) was stirred at 185° C. for 7 hours then cooled to room temperature. Ethanol (15 mL) was added and the resulting mixture was heated to dissolve as much solid as possible. Upon cooling to room temperature, the mixture was diluted with water and acidified to pH<7 with 1N HCl. The resulting mixture was stirred for 5 minutes and the product was filtered off and washed repeatedly with water. The resulting material was dried under reduced pressure to afford 2,3,5,6-tetradeutero-4-((4-hydroxypyrimidin-2-yl)amino)benzonitrile (7) as a brown solid (511 mg, 72%). MS (ESI) 217.1 [(M+H)⁺].

### Step 2. 2,3,5,6-Tetradeutero-4-((4-chloropyrimidin-2-yl)amino)benzonitrile (8)

POCl₃ (2 mL) was added to 7 (131 mg, 0.606 mmol) and the reaction was stirred at reflux for 30 minutes. The reaction was then cooled, poured slowly into ice-water and the resulting slurry was stirred for 3 hours. The product was removed by filtration and washed repeatedly with water. The resulting solid was dried under reduced pressure to afford 2,3,5,6-tetradeutero-4-((4-chloropyrimidin-2-yl)amino)benzonitrile (8) as a brown solid (83 mg, 58%). MS (ESI) 235.1 [(M+H)⁺].

### Step 3. 4-((4-((4-Bromo-3,5-dideutero-2,6-bis)trideuteromethyl)phenyl)amino)pyrimidin-2-yl)amino)-2,3,5,6-tetradeuterobenzonitrile (9a)

A mixture of chloropyrimidine 8 (83.0 mg, 0.354 mmol) and bromoaniline 6 (74.0 mg, 0.354 mmol) was stirred at 150° C. for 1 hour. The reaction was then cooled to room temperature, diluted with 10% K₂CO₃ and extracted with 5% MeOH/CH₂Cl₂ (3×50 mL). The organic layers were combined, dried (Na₂SO₄), filtered and concentrated. The resulting residue was purified via silica gel column chromatography (15-60% EtOAc/heptane) to afford 4-((4-((4-bromo-3,5-dideutero-2,6-bis(trideuteromethyl)phenyl)amino)pyrimidin-2-yl)amino)-2,3,5,6-tetradeuterobenzonitrile (9a) as a tan solid (71 mg, 49%). MS (ESI) 406.1 [(M+H)⁺].

### Step 4. (E)-2,3,5,6-Tetradeutero-4-((4-((3,5-dideutero-4-(1,2-dideutero-2-cyanovinyl)-2,6-bis(trideuteromethyl)phenyl)amino)pyrimidin-2-yl)amino)benzonitrile (Compound 244)

Nitrogen gas was bubbled through a solution of 9 (71.0 mg, 0.174 mmol) in acetonitrile (9 mL) for 15 minutes then acrylontrile-d3 (121 μL, 1.74 mmol, CDN Isotopes, 98 atom % D) was added followed by triethylamine (24.0 μL, 0.174 mmol), Pd(OAc)₂ (8.00 mg, 0.035 mmol) and P(o-tol)₃ (53.0 mg, 0.174 mmol). The reaction was stirred at 150° C. in a sealed tube for 15 hours then was cooled to room temperature, diluted with water and extracted with CH₂Cl₂ (3×25 mL). The organic layers were combined, dried (MgSO₄), filtered and concentrated to afford a brown oil containing (E)-2,3,5,6-tetradeutero-4-((4-((3,5-dideutero-4-(1,2-dideutero-2-cyanovinyl)-2,6-bis(trideuteromethyl)phenyl)-amino)pyrimidin-2-yl)amino)benzonitrile (244). MS (ESI) 381.2 [(M+H)⁺].

### Example 4

### Synthesis of (E)-4-((4-((3,5-d₂-4-(2-cyanovinyl)-2-cyanovinyl)-2,6-bis(d₃-methyl)phenyl)amino)pyrimidin-2-yl)amino)benzonitrile (Compound 201).

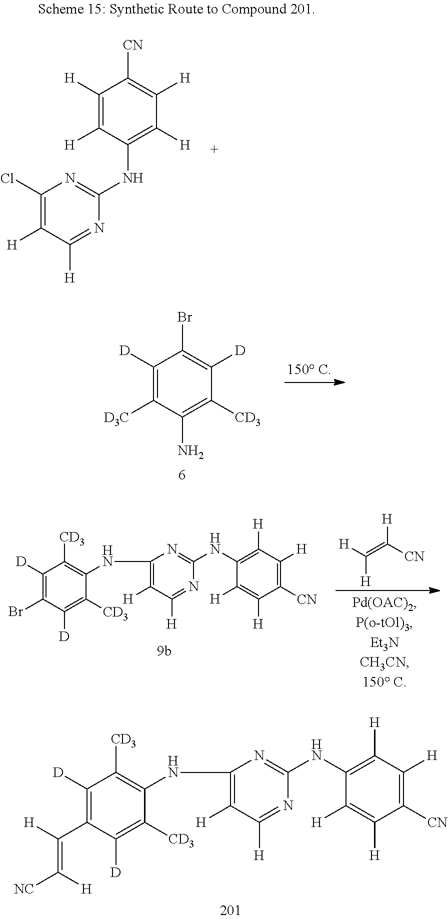

### Step 1. 4-((4-((4-Bromo-3,5-dideutero-2,6-bis(trideuteromethyl)phenyl)amino)pyrimidin-2-yl)amino)-benzonitrile (9b)

Prepared according to the procedure for 9a employing 4-((4-chloropyrimidin-2-yl)amino)benzonitrile (120 mg, 0.520 mmol, prepared according to the procedure described in: X.-Q. Feng et al. Chem. Med. Chem. 2009, 4, 219-224) and bromoaniline 6 (108 mg, 0.520 mmol) to afford 4-((4-((4-bromo-3,5-dideutero-2,6-bis(trideuteromethyl)-phenyl)amino)pyrimidin-2-yl)amino)-benzonitrile (9b) as a tan solid (97 mg, 46%). MS (ESI) 402.1 [(M+H)⁺].

### Step 2. (E)-4-((4-((3,5-dideutero-4-(2-cyanovinyl)-2,6-bis(trideuteromethyl)phenyl)amino)-pyrimidin-2-yl)amino)benzonitrile (201)

Prepared according to the procedure for 1 employing arylbromide 10 (97.0 mg, 0.241 mmol) and acrylonitrile (158 μL, 2.41 mmol) to afford a brown oil containing (E)-4-((4-((3,5-dideutero-4-(2-cyanovinyl)-2,6-bis(trideuteromethyl)phenyl)-amino)pyrimidin-2-yl)-amino)benzonitrile (2). MS (ESI) 375.2 [(M+H)⁺].

### Example 5

### Synthesis (E)-2,3,5,6-d₄-4-((4-((3,5-d₂-4-(2-cyanovinyl)-2,6-bis(d₃-methyl)phenyl)amino)pyrimidin-2-yl)amino)benzonitrile (Compound 219)

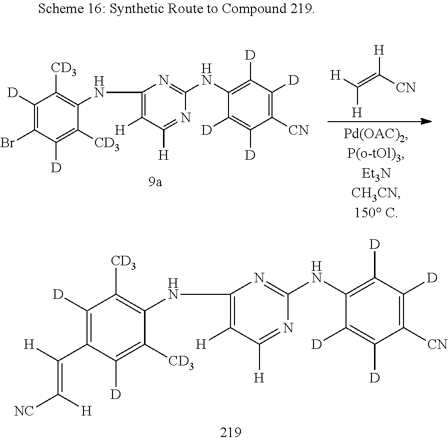

### (E)-2,3,5,6-Tetradeutero-4-((4-((3,5-dideutero-4-(2-cyanovinyl)-2,6-bis(trideutero-methyl)phenyl)amino)pyrimidin-2-yl)amino)benzonitrile (219)

Prepared according to the procedure for 1 employing arylbromide 9 (100 mg, 0.246 mmol) and acrylonitrile (162 μL, 2.46 mmol) to afford a brown oil containing (E)-2,3,5,6-tetradeutero-4-((4-((3,5-dideutero-4-(2-cyanovinyl)-2,6-bis(trideutero-methyl)phenyl)amino)pyrimidin-2-yl)amino)benzonitrile (3). MS (EST) 377.2 [(M−H)⁻].

### Example 6

### Evaluation of Metabolic Stability

Microsomal Assay:

Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability:

7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μl, of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-deuterated counterpart of the compound of Formula I or Formula VI and the positive control, 7-ethoxycoumarin (1 μM). Testing is done in triplicate.

Data Analysis:

The in vitro t_{1/2}s for test compounds are calculated from the slopes of the linear regression of % parent remaining (In) vs incubation time relationship.

in vitro t_{1/2}=0.693/k

k=−[slope of linear regression of % parent remaining(ln) vs incubation time]

Data analysis is performed using Microsoft Excel Software.

### Example 7

### Evaluation of Plasma Protein Binding

Determination of Plasma Protein Binding:

Stock solutions of test compounds (7.5 mM) are prepared in DMSO. Each stock solution is diluted to 1 μM in plasma from the appropriate species (human, monkey, dog, rat etc). Aliquots of plasma containing test compound (200 μL) are added to the donor chamber, and phosphate-buffered saline (PBS) buffer aliquots (350 μL) are added to the receiver chamber of a Rapid Equilibrium Dialysis Device (RED). The RED plate is incubated for 4-6 hours at 37° C. with orbital shaking. Incubations are performed in triplicate. After a 4-6 hour incubation period, a 50 μL aliquot of plasma from the donor chamber is mixed with 50 μL of the buffer and this mixture is treated with 200 mL of acetonitrile containing 50 ng/mL of internal standard (for bioanalysis). At the same time, a 50 μL aliquot of buffer from the receiver chamber is mixed with 50 μL of plasma and this mixture is treated with 200 μL of acetonitrile containing 50 ng/mL of internal standard (for bioanalysis). The acetonitrile-treated samples are centrifuged to pellet precipitated protein, and the supernatants are analyzed for parent (test compound) by LC-MS/MS. The metabolic stability in plasma and % recovery of the test compound are determined in parallel.

Data Analysis:

The percent of test compound bound to plasma proteins is calculated as shown below:

% Unbound test compound=(Peak area ratio in receiver chamber/Peak area ratio in donor chamber)×100

wherein the peak area ratio is given by the following relationship:

peak area ratio=peak area of test compound/peak area of internal standard

Peak areas are determined by LC-MS/MS.

The percentage of bound test compound is given by the following relationship:

Percentage of bound test compound=100%−percentage of unbound test compound

The metabolic stability of a test compound in plasma is calculated as shown below:

% test compound remaining in plasma after 4-6 hour incubation=[(Peak area ratio of test compound in plasma after incubation/Peak area ratio at 0 hr)]×100

The % recovery is calculated as shown below:

Recovery=[(Peak area ratio of test compound in receiver chamber at 4-6 hour+Peak area ratio in donor chamber at 4-6 hour)/(Peak area ratio in plasma at 0 hour)]×100

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound of Formula I 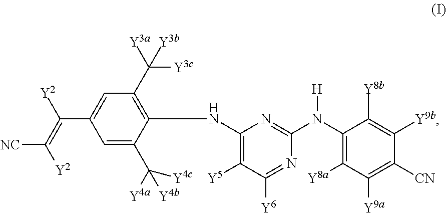
or a pharmaceutically acceptable salt thereof,
wherein each Y is independently hydrogen or deuterium,
provided that at least one Y is deuterium.

2. The compound of claim 1 wherein:
Y¹ and Y² are the same;
each of Y^{3a}, Y^{3b} and Y^{3c} is the same;
each of Y^{4a}, Y^{4b} and Y^{4c} is the same;
Y⁵ and Y⁶ are the same;
Y^{8a} and Y^{8b} are the same; and
Y^{9a} and Y^{9b} are the same.

3. The compound of claim 2 wherein each of Y^{3a}, Y^{3b} and Y^{3c} is hydrogen.

4. The compound of claim 2 wherein each of Y^{3a}, Y^{3b} and Y^{3a} is deuterium.

5. The compound of any one of claims 2-4, wherein each of Y^{4a}, Y^{4b} and Y^{4c} is hydrogen.

6. The compound of any one of claims 2-4, wherein each of Y^{4a}, Y^{4b} and Y^{4c} is deuterium.

7. The compound of claim 2, wherein Y¹ and Y² are each hydrogen and the compound is selected from any compound in the table below:

| | Each of | Each of | | | Each of | Each of |
|---|---|---|---|---|---|---|
| Cmpd | Y^{3a}, Y^{3b}, | Y^{4a}, Y^{4b}, | | | Y^{8a} and | Y^{9a} and |
| # | and Y^{3c} | and Y^{4c} | Y⁵ | Y⁶ | Y^{8b} | Y^{9b} |
| 100 | H | D | H | H | H | H |
| 101 | D | D | H | H | H | H |
| 102 | H | H | D | D | H | H |
| 103 | H | D | D | D | H | H |
| 104 | D | D | D | D | H | H |
| 105 | H | H | H | H | D | H |
| 106 | H | D | H | H | D | H |
| 107 | D | D | H | H | D | H |
| 108 | H | H | D | D | D | H |
| 109 | H | D | D | D | D | H |
| 110 | D | D | D | D | D | H |
| 111 | H | H | H | H | H | D |
| 112 | H | D | H | H | H | D |
| 113 | D | D | H | H | H | D |
| 114 | H | H | D | D | H | D |
| 115 | H | D | D | D | H | D |
| 116 | D | D | D | D | H | D |
| 117 | H | H | H | H | D | D |
| 118 | H | D | H | H | D | D |
| 119 | D | D | H | H | D | D |
| 120 | H | H | D | D | D | D |
| 121 | H | D | D | D | D | D |
| 122 | D | D | D | D | D | D |

or a pharmaceutically acceptable salt thereof.

8. The compound of claim 2, wherein Y¹ and Y² are each deuterium and the compound is selected from any compound in the table below:

| Cmpd | Each of | Each of | | | Each of | Each of |
|---|---|---|---|---|---|---|
| # | Y³ | Y⁴ | Y⁵ | Y⁶ | Y⁸ | Y⁹ |
| 125 | H | D | H | H | H | H |
| 126 | D | D | H | H | H | H |
| 127 | H | H | D | D | H | H |
| 128 | H | D | D | D | H | H |
| 129 | D | D | D | D | H | H |
| 130 | H | H | H | H | D | H |
| 131 | H | D | H | H | D | H |
| 132 | D | D | H | H | D | H |
| 133 | H | H | D | D | D | H |
| 134 | H | D | D | D | D | H |
| 135 | D | D | D | D | D | H |
| 136 | H | H | H | H | H | D |
| 137 | H | D | H | H | H | D |
| 138 | D | D | H | H | H | D |
| 139 | H | H | D | D | H | D |
| 140 | H | D | D | D | H | D |
| 141 | D | D | D | D | H | D |
| 142 | H | H | H | H | D | D |
| 143 | H | D | H | H | D | D |
| 144 | D | D | H | H | D | D |
| 145 | H | H | D | D | D | D |
| 146 | H | D | D | D | D | D |
| 147 | D | D | D | D | D | D |

or a pharmaceutically acceptable salt thereof.

9. A compound of Formula VI 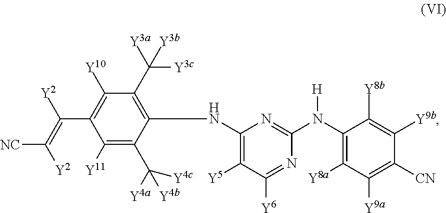
or a pharmaceutically acceptable salt thereof,
wherein each Y is independently hydrogen or deuterium,
provided that at least one Y is deuterium.

10. The compound of claim 9 wherein:
Y¹ and Y² are the same;
each of Y^{3a}, Y^{3b} and Y^{3c} is the same;
each of Y^{4a}, Y^{4b} and V^{4c} is the same;
Y⁵ and Y⁶ are the same;
Y^{8a} and Y^{8b} are the same;
Y^{9a} and Y^{9b} are the same; and
Y¹⁰ and Y¹¹ are the same.

11. The compound of claim 10, wherein Y¹ and Y² are each hydrogen, Y¹⁰ and Y¹¹ are each deuterium and the compound is selected from any compound in the table below:

| Cmpd | Each of | Each of | | | Each of | Each of |
|---|---|---|---|---|---|---|
| # | Y³ | Y⁴ | Y⁵ | Y⁶ | Y⁸ | Y⁹ |
| 200 | H | D | H | H | H | H |
| 201 | D | D | H | H | H | H |
| 202 | H | H | D | D | H | H |
| 203 | H | D | D | D | H | H |
| 204 | D | D | D | D | H | H |
| 205 | H | H | H | H | D | H |
| 206 | H | D | H | H | D | H |
| 207 | D | D | H | H | D | H |
| 208 | H | H | D | D | D | H |
| 209 | H | D | D | D | D | H |
| 210 | D | D | D | D | D | H |
| 211 | H | H | H | H | H | D |
| 212 | H | D | H | H | H | D |
| 213 | D | D | H | H | H | D |
| 214 | H | H | D | D | H | D |
| 215 | H | D | D | D | H | D |
| 216 | D | D | D | D | H | D |
| 217 | H | H | H | H | D | D |
| 218 | H | D | H | H | D | D |
| 219 | D | D | H | H | D | D |
| 220 | H | H | D | D | D | D |
| 221 | H | D | D | D | D | D |
| 222 | D | D | D | D | D | D |

or a pharmaceutically acceptable salt thereof.

12. The compound of claim 10, wherein Y¹ and Y² are each deuterium, Y¹⁰ and Y¹¹ are each deuterium and the compound is selected from any compound in the table below:

| Cmpd | Each of | Each of | | | Each of | Each of |
|---|---|---|---|---|---|---|
| # | Y³ | Y⁴ | Y⁵ | Y⁶ | Y⁸ | Y⁹ |
| 225 | H | D | H | H | H | H |
| 226 | D | D | H | H | H | H |
| 227 | H | H | D | D | H | H |
| 228 | H | D | D | D | H | H |
| 229 | D | D | D | D | H | H |
| 230 | H | H | H | H | D | H |
| 231 | H | D | H | H | D | H |
| 232 | D | D | H | H | D | H |
| 233 | H | H | D | D | D | H |
| 234 | H | D | D | D | D | H |
| 235 | D | D | D | D | D | H |
| 236 | H | H | H | H | H | D |
| 237 | H | D | H | H | H | D |
| 238 | D | D | H | H | H | D |
| 239 | H | H | D | D | H | D |
| 240 | H | D | D | D | H | D |
| 241 | D | D | D | D | H | D |
| 242 | H | H | H | H | D | D |
| 243 | H | D | H | H | D | D |
| 244 | D | D | H | H | D | D |
| 245 | H | H | D | D | D | D |
| 246 | H | D | D | D | D | D |
| 247 | D | D | D | D | D | D |

or a pharmaceutically acceptable salt thereof.

13. The compound of claim 1 or claim 9, wherein any atom not designated as deuterium is present at its natural isotopic abundance.

14. A pyrogen-free composition comprising an effective amount of a compound of claim 1 or claim 9 or a pharmaceutically acceptable salt thereof and a pharmaceutically acceptable carrier.

15. A method of inhibiting the conversion of single-stranded genomic RNA of HIV to double-stranded viral DNA in a cell, comprising contacting a cell with a compound of claim 1 or claim 9 or a pharmaceutically acceptable salt thereof.

16. A method of treating HIV infection in a subject, comprising administering to the subject in need thereof an effective amount of a composition of claim 14.
